(19) United States
(12) Patent Application Publication
(10) Pub. No.: US 2009/0062701 A1
(43) Pub. Date: Mar. 5, 2009

(54) LOWER EXTREMITY COMPRESSION DEVICES, SYSTEMS AND METHODS TO ENHANCE CIRCULATION

(75) Inventors: Demetris Yannopoulos, Saint Paul, MN (US); Keith Lurie, Minneapolis, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Advanced Circulatory Systems, Inc., Minneapolis, MN (US)

(21) Appl. No.: 12/165,366

(22) Filed: Jun. 30, 2008

Related U.S. Application Data
(60) Provisional application No. 60/947,346, filed on Jun. 29, 2007.

(51) Int. Cl. A61H 31/00; A61M 16/20

(52) U.S. Cl. 601/41; 128/200.24

(57) ABSTRACT

Systems and methods for enhancing circulation are described. In one particular embodiment, the invention provides a method for enhancing circulation. The method comprises attaching at least one compression device to at least a portion of a person's lower extremity. The person's chest is repetitively compressed so that the chest experiences a compression phase and a recoil phase or decompression. Also, the person's lower extremity is compressed using the compression device during at least some of the recoil phases.

### CROSS-REFERENCES TO RELATED APPLICATIONS

This application is a nonprovisional of, and claims the benefit of priority to, U.S. Provisional Patent Application No. 60/947,346 filed Jun. 29, 2007. This application is also related to U.S. patent application Ser. No. 12/119,374 filed May 12, 2008. The entire content of each of the above applications is incorporated herein by reference for all purposes.

### BACKGROUND OF THE INVENTION

Embodiments of the present invention relate generally to the field of circulatory enhancement, and in particular to systems and methods for increasing blood circulation, especially cardiac and cerebral perfusion in patients requiring or in need of treatment for a variety of conditions including cardiac arrest, shock of many etiologies (e.g. cardiogenic, hemorrhagic), heart failure, sepsis, blood loss, head injury, and stroke.

Millions of people suffer life-altering and life-threatening consequences from any of a variety of medical conditions and disease states that impair circulation. These medical conditions and disease states range from one-time occurrences to chronic conditions, and include shock, traumatic brain injury, cardiac arrest, dehydration, kidney failure, congestive heart failure, wound healing, diabetes, stroke, respiratory failure, and orthostatic hypotension. The consequences of reduced circulation are severe and burden the health care system with billions of dollars of expenditures on an annual basis.

Despite recent advances in the field of circulatory enhancement, the need for improved approaches for treating patients with impaired circulation remains an important medical challenge. For example, there is an ongoing need for non-invasive techniques that enhance circulation of blood throughout the body, thereby increasing the opportunity for survival and the quality of life of patients who experience major medical emergencies and severe circulatory conditions. There is a particular need for circulation enhancement techniques that can be used during or as part of a cardiopulmonary resuscitation. Embodiments of the present invention provide effective solutions to at least some of these needs.

### BRIEF SUMMARY OF THE INVENTION

Many individuals with low blood pressure have a need to increase their blood circulation to maintain adequate vital organ perfusion, especially brain perfusion. Persons who are in cardiac arrest and shock due to a variety of reasons including ventricular fibrillation, cardiac pump failure, trauma, dehydration, or sepsis suffer from dangerously low blood pressure and thus need to increase their blood circulation. Persons suffering from poor cerebral circulation due to head trauma or stroke, for example, would also benefit from an increase in cerebral perfusion pressure. Similarly, patents in cardiac arrest benefit from higher coronary perfusion pressures to help restart the heart and higher cerebral perfusion pressure to help maintain brain viability and avoid severe cerebral injury. During the treatment of cardiac arrest with cardiopulmonary resuscitation (CPR), an increase in vital organ blood flow is critical for survival. Despite current methods of CPR, most people die after cardiac arrest. One of the major reasons is that blood flow to the heart and brain is very poor with traditional manual closed chest CPR. Greater circulation of blood during CPR will result in improved outcomes.

Certain embodiments of the invention provide exemplary methods for increasing circulation during cardiac arrest and CPR and other states of low blood pressure. In one aspect, one or more circumferential cuffs are placed around a portion of the lower extremities, such as the upper and/or lower parts of the lower extremities. The cuffs are configured to rapidly compression and release, thereby squeezing the lower extremities on a regular and repetitive basis. The lower extremity compressions are timed to occur with each chest wall recoil, or after a certain number of chest wall recoil cycles. For patients who are not in cardiac arrest, the lower extremity compressions may be timed to occur between each cardiac contraction or after a certain number of cardiac contractions. The devices and methods may use a means to communicate with the chest compression device and lower extremity compression cuffs. This approach may optionally be used during CPR with an impedance threshold device (ITD) or an intrathoracic pressure regulator (ITPR), to reduce the rise in intracranial pressure and right atrial pressure associated with the transfer of blood back to the right heart.

The methods and devices can be used with any method of CPR that involves compressing the chest or the sternum. Further, it can be used for several purposes. For example, the thigh compression cuffs can also be used as a means to cool the lower extremities, thus promoting hypothermia at the same time as circulation. Hypothermia improves outcomes in patients in cardiac arrest. For example, interposed lower extremity compressions during CPR augments diastolic coronary artery pressure and cerebral perfusion pressure by >10 mmHg. This improves the likelihood of survival after cardiac arrest. With the methods and devices, the lower extremities and buttocks regions can be compressed but efforts may need to be taken to minimally compress the abdomen. Abdominal compression can result in a rise in intrathoracic pressure and more importantly in intracranial pressure, (via direct pressure transfer through to the spinal cord), that can be particularly harmful, since this elevation in intracranial pressure increases the resistance to forward cerebral blood flow. By contrast, compression of the lower extremities, that are not contiguous to the spinal cord, does not result in an increase in intrathoracic pressure. A 1:1 ratio between chest compression and extremity compression improves circulation, but other ratios are also of potential benefit.

In one particular embodiment, the invention provides a method for enhancing circulation. The method comprises attaching at least one compression device to at least a portion of a person's lower extremity. The person's chest is repetitively compressed so that the chest experiences a compression phase and a recoil phase or decompression. Also, the person's lower extremity is compressed using the compression device during at least some of the recoil phases.

In one aspect, the chest is compressed using a circumferential vest. In some cases, a valve system is coupled to the person's airway. The valve system is configured to prevent respiratory gas flow to the person's lungs during the decompression phase until a negative airway pressure achieved equals the opening pressure of the valve system. Optionally, air may be withdrawn from the person's lungs using a vacuum source until a certain negative intrathoracic pressure is achieved. In one example, the opening pressure of the valve system is between about −7 cm H₂O and about −20 cm H₂O.

The invention further provides a system for enhancing circulation. The system comprises a chest wall compression/decompression mechanism to repeatedly compress and decompress the patient's chest. At least one lower extremity compression device is provided and is adapted to be coupled to at least a portion of a person's lower extremity. Also, a controller is used to control actuation of the chest wall compression/decompression mechanism and the lower extremity compression device such that the lower extremity compression device compresses the person's lower extremity following at least some of the chest wall compressions.

In one aspect, a valve system is provided as is adapted to be coupled to the person's airway. The valve system is configured to prevent respiratory gas flow to the person's lungs during decompression until a negative airway pressure achieved equals the opening pressure of the valve system. The opening pressure of the valve system may be between about −7 cm H₂O and about −20 cm H₂O.

In another aspect, embodiments of the present invention encompass methods for administering circulation treatment to a person. Methods can include contacting a compression device with at least a portion of a lower extremity of the person, repetitively compressing the person's chest so that the chest experiences a compression phase and a decompression phase, and compressing the portion of the lower extremity using the compression device during at least a portion of the decompression phase. In some cases, a lower extremity compression is timed to occur with each chest wall recoil. In some cases, a lower extremity compression is timed to occur after a certain number of chest wall recoil cycles. Embodiments encompass methods where the person is in cardiac arrest. According to some embodiments, methods may include cooling at least a second portion of the lower extremity with the compression device. Methods may also include interfacing a valve system to a person's airway, where the valve system having a threshold valve and an airway member that interfaces with the person's airway, and threshold valve is configured to prevent or impede respiratory gas flow to the person's lungs during a portion of an inhalation event until the inspiration equals or exceeds an opening pressure of the threshold valve. Methods may also involve supplying a small level of external vacuum at a location downstream of the threshold valve. In some embodiments, the chest is compressed using a compression vest. In some embodiments, the chest is compressed using an automated compression apparatus. In some embodiments, the chest is compressed using an active compression decompression device. Methods may further include sensing at least one physiological parameter of the person. According to some methods, a chest compression parameter is determined based on the sensed physiological parameter. According to some methods, a lower extremity compression parameter is determined based on the sensed physiological parameter. Optionally, methods may include coordinating a chest compression and a lower extremity compression based on the sensed physiological parameter.

In another aspect, embodiments encompass devices for administering cardiopulmonary resuscitation to or treating low circulation in a patient. Devices may include a chest compression pad configured to compress the chest, a chest decompression mechanism configured to decompress the chest either passively or actively, a signal processor configured to record a sensed physiological parameter, and a lower extremity compression device that periodically compresses a lower extremity in a coordinated manner with a chest compression, a chest decompression, or both. In some cases, devices may include a power source that drives the chest compression pad, the chest decompression mechanism, and the lower extremity compression device, and that is coupled with a means to coordinate compressions and decompressions of the chest and the lower extremities.

In still another aspect, embodiments of the present invention encompass methods for treating a patient suffering from or experiencing cardiac arrest or low blood circulation. Methods may include providing to the patient a periodic positive pressure ventilation and generating a negative airway pressure when not providing the positive pressure ventilation from −3 to −15 mmHg, actively compressing and either actively or passively decompressing the patient's chest after each compression, and compressing a lower extremity of the patient in a synchronized manner with the chest compressions based upon a timing device or a measured physiological parameter.

Embodiments also encompass devices to treat a patient in cardiac arrest or lower blood circulation that include a means to provide periodic positive pressure ventilation and generate a negative airway pressure when not providing positive pressure ventilation from −3 to −15 mmHg, a means to actively compress and either actively or passively decompress the chest after each compression, and a means to compress the lower extremities in a synchronized manner with the chest compressions based upon a timing device or a measured physiological parameter.

For a fuller understanding of the nature and advantages of the present invention, reference should be had to the ensuing detailed description taken in conjunction with the accompanying drawings.

### BRIEF DESCRIPTION OF THE DRAWINGS

FIG. 1 illustrates aspects of a circulatory treatment system according to embodiments of the present invention.

FIG. 2 illustrates aspects of a circulatory treatment system according to embodiments of the present invention.

FIG. 3 illustrates aspects of a circulatory treatment system according to embodiments of the present invention.

FIG. 4 illustrates aspects of a circulatory treatment system according to embodiments of the present invention.

FIG. 5 illustrates aspects of a circulatory treatment system according to embodiments of the present invention.

FIG. 6 illustrates aspects of a circulatory treatment system according to embodiments of the present invention.

FIG. 7 illustrates aspects of a circulatory treatment method according to embodiments of the present invention.

### DETAILED DESCRIPTION OF THE INVENTION

This invention describes methods and devices for increasing circulation in low blood flow states, including CPR, by promoting blood flow from the lower extremities into the heart after each chest compression. The methods and devices include a way to compress the lower extremities and optionally the buttocks in a manner that is timed with the decompression or chest wall recoil phase of CPR. During each subsequent compression, the blood is pushed out of the heart into the arterial circulation. In this manner, circulation is enhanced during CPR as more blood is transferred from the lower extremities to the heart during the chest wall recoil phase to better preload the heart for the subsequent compression. The lower extremities can be compressed on a one to one ratio with chest compressions, or at different ratios. For example, the lower extremities could be compressed prior to each chest compression, prior two every other chest compression, prior to every third chest compression and the like. The lower extremities can include the arms, legs and/or buttocks and may be compressed in a variety of ways. This may be done by applying circumferential pressure (with a circumferential pressure cuff) and/or “directly” (by physically pushing on the top of the thighs) and/or by applying some other form of pressure.

Embodiments of the present invention encompass systems and methods for enhancing circulation in a patient. These techniques are well suited for use in treating individuals that may suffer from or are at risk of developing a variety of clinical conditions due to low blood flow. For example, exemplary devices and methods can be used to treat subjects presenting sudden cardiac arrest, traumatic injury, heat stroke, fainting, and the like, which can result in or from states of low blood flow or perfusion. In such cases, a lack of adequate blood flow back to the heart can contribute to the low blood pressure. States of low blood flow can impair the body's circulatory function, which delivers oxygen to the body's vital organs and removes toxic cellular waste. Circulatory enhancement techniques disclosed herein can use compression techniques increase blood flow to the body's vital organs. Further, such approaches can enhance the body's biophysical performance without depending upon pharmaceutical or other outside agents. In many cases, these systems and methods can be used in patients receiving CPR to increase venous blood return to the heart.

Multiple methods of chest compression can be used, including any currently existing techniques used to compress the chest during CPR when patients are in cardiac arrest. In this life-threatening situation, the heart is not capable of circulating blood so non-invasive external means are used to assist in the circulation of blood to the vital organs including the heart, lungs, and brain. Such techniques to circulate blood during cardiac arrest include manual closed chest CPR, active compression decompression (ACD or ACDC) CPR, mechanical CPR with manual or automated devices that compress the chest either on the sternum, circumferentially, or both, and either allow the chest to recoil passively or actively, devices that compress the chest wall and then function like an iron lung and actively expand the thoracic cage and the like.

Another optional way to increase circulation during CPR and other states of low blood pressure where cardiac preload is deficient is to use an impedance threshold device (ITD) or an intrathoracic pressure regulator (ITPR) to lower intrathoracic pressure during the chest wall recoil phase of CPR, thereby enhancing the transfer of blood from outside the thorax into the right heart. Using this approach, the intrathoracic pressure is lower during the chest wall recoil phase of CPR and blood is drawn back to the heart, filling the pump prior to the next compression. In addition, in the setting of ongoing chest compressions the ITD works by impedes or preventing respiratory gases from reentering the lungs, except when a breath is delivered by the rescuer, thus there is a net loss of air/O2 volume in the lungs which provides more space for blood that is drawn in from the non-thoracic space.

Embodiments of the present invention can be particularly effective when used with an ITD or ITPR during CPR. By generating a greater negative intrathoracic vacuum with each chest wall recoil, it is possible to transfer significant volumes of blood back to the heart by sequential lower extremity compressions. This results in greater cardiac output and great blood flow to the brain. Examples of such ITDs and ITPRs are described in U.S. Pat. Nos. 5,692,498; 6,062,219; 6,526,973; and 6,604,523, the complete disclosures of which are herein incorporated by reference. Relatedly, any of a variety of impeding or preventing mechanisms may be used to prevent or impede respiratory gases from flowing back into the lungs, including those described in U.S. Pat. Nos. 5,551,420; 5,692,498; 6,062,219; 5,730,122; 6,155,257; 6,234,916; 6,224,562; 6,986,349; and 7,204,251, the complete disclosures of which are herein incorporated by reference. The mechanisms may be configured to completely prevent or provide resistance to the inflow of respiratory gases into the patient while the patient inspires. In devices that completely prevent the flow of respiratory gases, the valves may be configured as pressure responsive valves that open after a threshold negative intrathoracic pressure has been reached. Such systems and devices can be referred to herein collectively by the name “impedance threshold device” or “ITD”. However, it will be appreciated that a wide variety of devices may be used. As another example, devices may be interfaced with a person's airway to prevent respiratory gas flow to the person's lungs during a portion of an inhalation event to enhance circulation and decrease intracranial pressure, including those described in U.S. Pat. No. 7,195,012, incorporated herein by reference. Valve systems according to embodiments of the present invention may incorporate features of these ITD or ITPR valves. Such valve systems can enhance circulation by prolonging the duration and increasing the magnitude of negative intrathoracic pressure in the chest to increase venous return. By enhancing the amount of venous blood flow into the heart and lungs, cardiopulmonary circulation is increased. The intracranial pressure is decreased by facilitating the flow of cerebral spinal fluid from the head to the spinal cord and by lowering the intrathoracic pressures during inhalation to repetitively lower pressure in the venous blood vessels out of the head jugular and vertebral veins) to facilitate venous blood flow out of the head. In some embodiments, valve systems may include aspects of vacuum supplemented check valves as described in U.S. patent application Ser. No. 12/141,864 filed Jun. 18, 2008, incorporated herein by reference. Such valve systems may include a threshold valve that prevents or impedes respiratory gases from flowing to the lungs until a certain amount of negative intrathoracic pressure (ITP), optionally in combination with or as supplemented by an external vacuum, is reached. In some cases, the opening pressure of the valve system may be between about −7 cm H₂O and about −25 cm H₂O. In some cases, the external vacuum level is less than the opening pressure of the valve system and is between about −1 cm H₂O and about −24 cm H₂O.

Methods and devices, such as ITDs that reduce the amount of respiratory gases inside the thorax by preventing said gases from reentering the thorax during the chest wall recoil phase, or by actively removing said gases either intermittently or continuously, result in less and less air in the thorax. Less air in the thorax makes room for more and more blood to return to the heart during the chest wall recoil phase. Application of the aforesaid m methods and devices cause a reduction in intrathoracic pressures, either during the chest wall recoil phase or continuously during the chest compression and decompression phases, which results in a simultaneous decrease in intracranial pressures. As such, application of these methods and devices increases circulation to the coronary arteries during the chest wall decompression phase, and increases blood flow to the brain during the compression and decompression phases, thereby delivering more oxygen-rich blood to the brain.

In certain specific embodiments, the invention provides methods and systems to enhance circulation, especially cerebral perfusion, in a person by utilizing one or more of the following devices/concepts which may be combined to act synergistically. One such device is a circumferential vest device that may include a metallic rim. The vest device is placed around the chest and is used as a base to generate active decompression and compression. One role of the vest is to generate the compression force to generate forward blood flow and to actively pull up the chest wall to enhance decompression and allow for full or overshoot recoil. As an alternative, standard CPR could be used instead of the vest. Also, ACD CPR could be used with a variety of other peripheral devices as described in U.S. Pat. Nos. 6,463,327; 6,587,726; 7,195,013 and copending U.S. application Ser. No. 11/679,693, filed Feb. 2, 2007, incorporated herein by reference.

Another device used to enhance circulation is a valve system that may be interfaced to a person's airway. The valve system is configured to prevent respiratory gas flow to the person's lungs during the decompression phase until a negative airway pressure achieved equals the opening pressure of the valve system (ITD). Alternatively, the valve system may be used to withdraw air from the lungs via and active vacuum source until a negative airway pressure is achieved (ITPR). In one arrangement, the opening pressure of the valve system is between about −7 cm H₂O and about −20 cm H₂O. The valve system can be made of a check valve, spring valve, duck valve, other mechanical one-way valves.

In one particular arrangement, the devices employed to compress the lower extremities comprise lower extremity, counter-pulsation, gas inflated cuffs, fitted around a portion of the thighs or the entire lower body, which are triggered by the decompression phase of CPR. The lower extremity device accomplishes two main objectives. First, it acts to enhance venous return and add to the effect of the valve system (ITD or ITPR) without imposing significant abdominal pressure increases that would normally lead to a rise in ICP. Second, it enhances diastolic pressure, increases coronary perfusion pressure and shifts more blood to the brain since the resistance in the lower extremities will always be kept above the systemic resistance of the cerebral circulation.

In some cases, addition of a gas (i.e., a mixture of nitrogen and atmospheric air) coolant can be used both as the primary source of inflation and as a direct surface cooling method while performing CPR. Also, in some embodiments, a sensor mechanism may be positioned at the chest to trigger the lower extremity counterpulsations, either based on the decompression timing (i.e., during CPR) or during the organized cardiac diastole with a normal beating heart. The thoracic sensor can be used to time with the QRS of the electrocardiogram, a thoracic compression during the election period of the failing heart and trigger the lower extremity counterpulsation during diastole of the beating heart.

By compressing the extremities during the chest wall decompression phase, diastolic arterial pressures are increased but arterial afterload is minimized during the chest compression phase. These combined actions results in a net increase in both coronary and cerebral perfusion.

Another aspect of this invention may include the addition of intravenous cooling, for example with ice cold saline to reach a cerebral temperature of 33 degrees C. within 4-5 minutes.

Method and system embodiments disclosed herein are well suited for use with patients suffering cardiac arrest as a result of myocardial infarction, anaphylactic shock, accidental hypothermia, pulmonary emboli, and the like. In some cases, the cause of cardiac arrest may be unknown. Embodiments may also be used with patients during routine medical procedures, such as surgery, catheterization, and the like.

Embodiments of the present invention encompass systems and methods that involve manual closed chest CPR, ACDC, mechanical CPR with manual or automated devices that compress the chest and either allow the chest to recoil passively or actively, and devices that compress the chest wall and then function like an iron lung and actively expand the thoracic cage. Some of these approaches and devices only compress the anterior aspect of the chest, such as the sternum, while other approaches and devices compress all or part of the thorax circumferentially. Some approaches and devices also compress the thorax and abdomen in an alternating sequence. Some approaches also involve compressing the lower extremities to enhance venous blood flow back to the heart and augment arterial pressure, so that more blood goes to the brain. Other approaches also involve compressing the back while the patient is lying on his/her stomach. Some devices include the non-invasive methods and devices outlined herein that are coupled with invasive devices, such as an intra-aortic balloon, and devices to simultaneously cool the patient.

Because the cardiac valves typically remain intact during CPR, blood is pushed out of the heart into the aorta during the chest compression phase of CPR. When the chest wall recoils, blood from extrathoracic compartments (e.g., the abdomen, upper limbs, and head) enters the thorax, specifically the heart and lungs. During the chest wall recoil phase, blood fills the cardiac chambers as well as the coronary arteries, i.e., the arteries that provide blood to the heart muscle. Blood flows to the brain during both the chest compression and decompression phases. The amount of blood flow to the brain depends upon the gradient between forward blood flow (determined in large part by the arterial pressure) and the resistance in flow into the brain (determined in large part by the intracranial pressure).

During the compression phase of closed chest manual (standard) CPR, air is pushed out of the thorax and into the atmosphere via the trachea and airways. During the decompression phase, air passively returns back into the thorax via the same airway system. As such, respiratory gases move out of and back into the thorax. With each compression the pressure within the chest is nearly instantaneously transmitted to the heart, and also to the brain via the spinal column and vascular connections. Thus, with each external chest compression, pressure is increased in the thorax and within all of the organs in the thorax.

Embodiments of the present invention can provide a systems-based approach that is optimized to interface with the patient's airway, provide the benefits of ITD therapy and maximize circulation to the heart and brain by compressing and decompressing the chest. Such techniques can provide an overall increase in the likelihood of a positive outcome after cardiac arrest.

Traditional or standard CPR also includes the delivery of a positive pressure breath periodically, in order to inflate the lungs and provide oxygen (“O₂”). In addition, positive pressure ventilation can provide a means to remove carbon dioxide (“CO₂”) from the lungs. Since the delivery of O₂ can be an important aspect of CPR, periodic positive pressure ventilation is traditionally delivered to inflate the lungs and provide oxygen.

Embodiments of the present invention encompass techniques that do not include positive pressure ventilation, that provide a means to lower intrathoracic pressure during CPR (to augment venous blood flow back to the heart and lower intracranial pressures), and that provide a means to prevent lung collapse. Accordingly, embodiments can optimize circulation and respiration during CPR while avoiding harmful effects of positive pressure ventilation.

Exemplary methods may include interfacing an airway system with a patient's airway, wherein the airway system includes at least a first lumen and a second lumen; repeatedly performing or administering CPR chest compressions on the patient; and simultaneously with the CPR chest compressions applying a continuous vacuum to the first lumen for a period of time ranging from 15 seconds to the end of the CPR chest compressions at a level sufficient to maintain a continual negative intrathoracic pressure in the patient, and injecting an effective volume of oxygen gas into the person's lungs at high velocity through the second lumen.

As used herein, including the appended claims, the “patient” can encompass any subject undergoing cardiopulmonary respiration (CPR), and may include both human and non-human animals.

As used herein including the appended claims, the phrase “airway system” is intended to include any system that is adapted to be interfaced with a patient's airway and has at least one lumen adapted to ventilate the patient's lungs during CPR; e.g. is adapted to move respiratory gases into and out of the patient's lungs. Such airway systems are sometimes referred to herein as “airway adjuncts” or “ventilation tubes”. Non-limiting examples of airway systems may include endrotracheal tubes, supraglottic airway devices, Combitubes, obturator airways, laryngeal mask airways, and the like. Airway systems of the present invention can also include at least a second lumen adapted to deliver oxygen gas into the patient's lungs.

As used herein including the appended claims, the phrase “CPR chest compressions” is intended to include any of the aforementioned CPR methods having a chest compression phase and a chest decompression (or recoil) phase. The chest compression phase serves to increase intrathoracic pressure and, thus, generate a pressure gradient between the thorax and the rest of the body, which in turn forces blood to the brain and other extra-thoracic organs. In addition, the chest compression phase causes the collapse of some of the bronchioles and, as a result, gas that is trapped in the distal portions of the airways is compressed. Thus, when there are respiratory gases in the lungs, the chest compression phase can help to open up the lungs and thus prevent atelectasis (collapse of the lungs). CPR chest compressions may also help to adequately exchange respiratory gases and help to maintain blood flow, as long as the lungs are partially inflated during the chest decompression phase. As a result, tissue oxygenation is maintained at a high level, CO₂ can be removed, and blood can move from the right heart to the left heart with a better match between perfusion and ventilation. In the context of the present invention, CPR chest compressions may be viewed as providing a motor, and the combination of continuous high velocity O₂-rich gas delivery and the maintenance of a continuous application of a continuous vacuum to the patient's airway negative intrathoracic pressure may be viewed as optimizing or improving the blood circulation to the heart and brain that is produced by that motor. In addition, the present invention may optimize the delivery of O₂ to and CO₂ removal from the patient's lungs.

The CPR chest compressions may also generate decompression phase negative intrathoracic pressure with each chest wall recoil. An ITD may be used to prevent or impede respiratory gases from returning to the thorax during the chest wall recoil of the decompression phase of each CPR chest compression. By preventing or impeding respiratory gases from reentering the lungs during the decompression phase of CPR, the ITD helps maintain the decompression phase negative intrathoracic pressure. However, even when an ITD is used, the level of decompression phase negative intrathoracic pressure during standard CPR may oscillate with each compression and decompression cycle.

As used herein, the phrase “continuous or continual negative intrathoracic pressure or vacuum” can mean a state in which, when simultaneously combined with CPR chest compressions and the optional injection of high velocity O₂, the application of vacuum to the patient's airway is not interrupted for a period of time ranging from 15 seconds to the end of the CPR chest compressions. In some cases it could be for at least 15 seconds and in other cases at least 30 seconds to the end of performing CPR. Pressure within the thorax may continually remain below atmospheric pressure, e.g. the intrathoracic pressure values may be continually negative relative to atmospheric pressure. Negative pressure may also be sometimes referred to herein as a vacuum.

In one embodiment of the invention, a continuous vacuum is applied to the patient's airway at a level sufficient to maintain or supplement a continual negative intrathoracic pressure during a compression phase. In some embodiments, a continuous vacuum may be applied to the patient's airway by connecting a vacuum source to the lumen of an airway system such as an endrotracheal tube. In other embodiments, the continuous vacuum may be applied to the patient's airway by other means; e.g. through a connector for the vacuum source at a remote location in a ventilation circuit or through a separate lumen, such as a nasal tube. As described above, values of the negative intrathoracic pressure provided by the continuous vacuum may oscillate; e.g. with each CPR chest compression, and therefore the intrathoracic pressures values may not remain continuously negative relative to atmospheric pressure. However, it is understood that the negative pressure (vacuum) applied to the patient's airway can remain continuously negative for at least 15 seconds, for example, during the performance of CPR chest compressions and the injection of high velocity O₂.

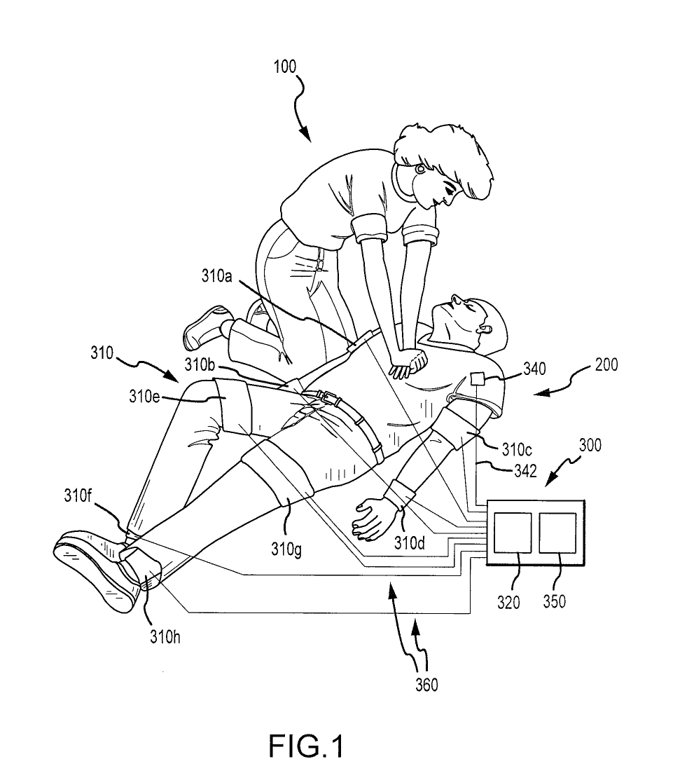

Turning now to the drawings, FIG. 1 illustrates a person or rescuer 100 administering a manual cardiopulmonary resuscitation chest compression to a patient 200 who is wearing one or more compression mechanisms or cuffs 310 of a compression system 300. As depicted here, an upper right arm cuff 310a, a lower right arm cuff 310b, an upper left arm cuff 310c, a lower left arm cuff 310d, an upper right leg cuff 310e, a lower right leg cuff 310f, an upper left leg cuff 310g, or a lower left leg cuff 310h, or any combination thereof, may be placed on the patient. Compression system 300 also include a computer or processor 320 that can control operation of the compression mechanisms 310. Optionally, compression system 300 may include a compression regulator 350 that regulates compression administered by compression mechanisms 310, via one or more compression mechanism lines 360. For example, a compression regulator 350 may include a pressure modulation mechanism that is configured to delivery varying amounts of pressure to the patient's extremities via inflatable cuffs which are attached to the pressure mechanism via pressure lines or tubes. Operation of compression regulator 350 may be controlled by processor 320. Compression mechanisms 310 can include any of a variety of devices that apply compression to a portion of the patients' body. Exemplary compression mechanisms include inflatable cuffs that are operated by way of fluid power or pneumatics. Compression mechanisms may optionally include mechanically actuated bands or pads. In some cases, a compression mechanism provides compression about the entire circumference of the patient's body or limb. In some cases, a compression mechanism provides compression about a portion of the circumference of the patient's body or limb.

In an exemplary procedure, the administrator or person providing CPR can place one or more cuffs on one or more of the patient's limbs. The administrator can deliver compressions to the chest of the patient, and the system can deliver compressions to the patient via the compression mechanisms. For example, the administrator or rescuer may apply a series of chest compressions to the patient and, in between the manual chest compressions, the system 300 can deliver compressions to one or more of the patient's limbs. In some embodiments, CPR can also involve intermittently providing positive pressure ventilation. Each time the chest is compressed and then allowed to recoil, blood circulates to the heart and brain. Because the heart may not be capable of circulating blood on its own, a non-invasive external means or mechanism can used to assist in the circulation of blood to the vital organs, including the heart, lungs, and brain. Methods and devices may also be used to circulate blood during cardiac arrest that manipulate of one or more of a patient's body parts, usually the chest, to increase the magnitude and duration of the patient's negative intrathoracic pressure.

Compression treatment system 300 may include one or more sensors 340 that can detect physiological parameters of the patient. Sensors can be configured to detect physiological parameters such as heart rate, respiration rate, temperature, blood gas (e.g. O₂, CO₂) concentration, blood pressure, blood flow, respiration gas (e.g. O₂, CO₂) concentration, cardiac output, intrathoracic pressure, intrathoracic volume, cardiac electrical activity, and the like. Processor 320 of compression system can be configured to receive signals from sensors 340, optionally via a sensor line 342, and process the signals according to desired processing methods. Processor 320 may also be configured to transmit operating signals or data to one or more compression mechanisms of compression system 300. In some cases, operating or controlling signals that control operation of the compression mechanisms can be based on signals or data received from sensors 340. In this way, processor 320 can provide a feedback technique, whereby physiological information is sensed from the patient, and a compression treatment is applied or administered to the patient based at least in part in response to one or more aspects of the physiological information. According to some embodiments, processor 320 can generate a compression regime or protocol that includes one or more treatment parameters, such as pressure, duration, timing, and the like. In some cases, the compression regime includes treatment parameters for lower extremity or limb compressions, and optionally includes treatment parameters for chest compressions. For example, processor 320 can generate a compression protocol that involves applying compressions to one or more lower extremities of a patient during a recoil or decompression phase.

Exemplary patient treatment protocols may therefore include processes as determined at least in part by a computer or processor having hardware, software, and/or firmware. Aspects of various method steps may be performed by modules, and the modules may comprise any of a wide variety of digital and/or analog data processing hardware and/or software arranged to perform or determine the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

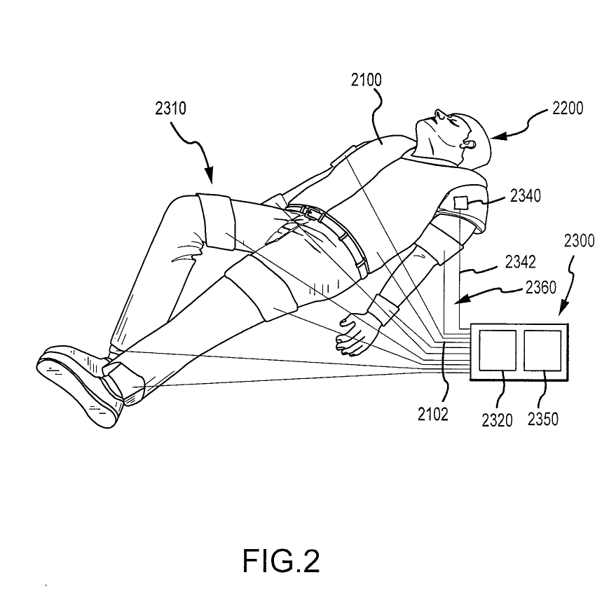

FIG. 2 shows an exemplary compression system 2300 that provides compression to one or more limbs or lower extremities of patient 2200. As shown here, patient 2200 may be provided with a compression vest 2100 that delivers chest compressions to the patient. Compression system 2300 may include a computer or processor 2320 that can control operation of the compression mechanisms or cuffs 2310. Optionally, processor 2320 may control operation of the compression vest 2100, for example via compression vest line 2102.

Compression treatment system 2300 may include one or more sensors 2340 that can detect physiological parameters of the patient. Sensors can be configured to detect physiological parameters such as heart rate, respiration rate, temperature, blood gas (e.g. O₂, CO₂) concentration, blood pressure, blood flow, respiration gas (e.g. O₂, CO₂) concentration, cardiac output, intrathoracic pressure, intrathoracic volume, cardiac electrical activity, and the like. Processor 2320 of compression system can be configured to receive signals from sensors 2340, optionally via sensor lines 2342, and process the signals according to desired processing methods. Processor 2320 may also be configured to transmit operating signals or data to one or more compression mechanisms of compression system 2300. In some cases, operating or controlling signals can be based on signals or data received from sensors 2340. In this way, processor 2320 can provide a feedback technique, whereby physiological information is sensed from the patient, and a compression treatment is applied or administered to the patient based at least in part on one or more aspects of the physiological information. According to some embodiments, processor 2320 can generate a compression regime or protocol that includes one or more treatment parameters, such as pressure, duration, timing, and the like. In some cases, the compression regime includes treatment parameters for lower extremity or limb compressions, and optionally includes treatment parameters for chest compressions. For example, processor 2320 can generate a compression protocol that involves applying compressions to one or more lower extremities of a patient during a recoil or decompression phase. The compression protocol can be administered at least in part by a compression regulator 2350 of system 2300, optionally via compression mechanism lines 2360.

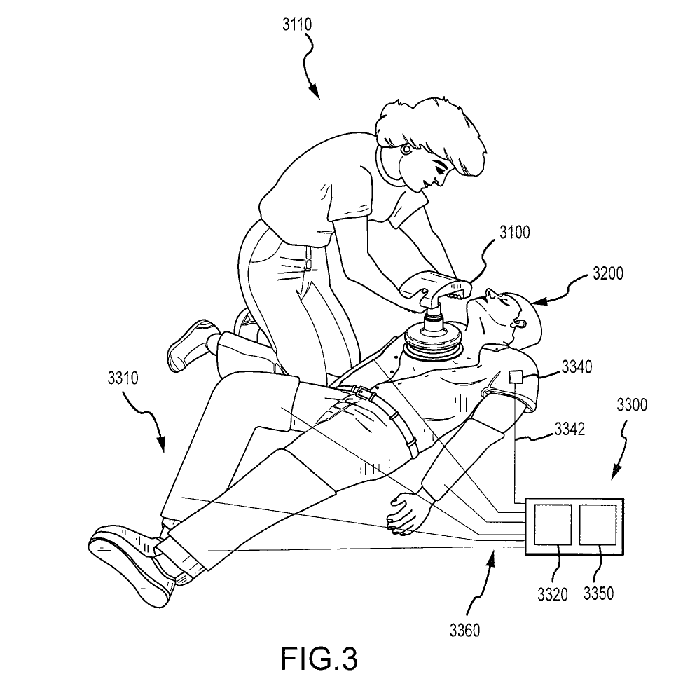

FIG. 3 depicts a compression system 3300 according to embodiments of the present invention. Compression system 3300 can deliver compression to one or more limbs or lower extremities of patient 3200 via compression mechanisms 3310. An operator or rescuer 3110 can apply a hand held Active Compression DeCompression (ACDC) device 3100 to the patient's body. Exemplary ACDC devices include those such as the Ambu CardioPump® (Ambu International, Glostrup, Denmark). ACDC device 3100 includes a suction element, such as an elastomeric cup, that can be placed against the patient's chest. In use, the suction element allows the operator to apply compressions to the chest, and also allows the operator to pull up or lift the chest to create decompression, resulting in additional amounts of negative pressure in the thorax, thereby facilitating the process of filling the heart with blood. ACDC devices may include a pressure gauge that determines, and optionally displays, the amount of compression or decompression administered to the patient. Compression system 3300 may include a computer or processor 3320 that can control operation of the compression mechanisms. Optionally, processor 3320 may send signals to or receive signals from ACDC device 3100. In some cases, such signals may include information that reflects the amount of compression or decompression administered to the patient.

Compression treatment system 3300 may include one or more sensors 3340 that can detect physiological parameters of the patient. Sensors can be configured to detect physiological parameters such as heart rate, respiration rate, temperature, blood gas (e.g. O₂, CO₂) concentration, blood pressure, blood flow, respiration gas (e.g. O₂, CO₂) concentration, cardiac output, intrathoracic pressure, intrathoracic volume, cardiac electrical activity, and the like. Processor 3320 of compression system can be configured to receive signals from sensors 3340, optionally via sensor line 3432, and process the signals according to desired processing methods. Processor 3320 may also be configured to transmit operating signals or data to one or more compression mechanisms of compression system 3300. In some cases, operating or controlling signals can be based on signals or data received from sensors 3340. In this way, processor 3320 can provide a feedback technique, whereby physiological information is sensed from the patient, and a compression treatment is applied or administered to the patient based at least in part on one or more aspects of the physiological information. According to some embodiments, processor 3320 can generate a compression regime or protocol that includes one or more treatment parameters, such as pressure, duration, timing, and the like. In some cases, the compression regime includes treatment parameters for lower extremity or limb compressions, and optionally includes treatment parameters for chest compressions. For example, processor 3320 can generate a compression protocol that involves applying compressions to one or more lower extremities of a patient during a recoil or decompression phase. Optionally, processor 3320 can generate a compression protocol that involves applying compressions to the patient's chest. In some cases, processor 3320 generate synchronized compressions of the chest and one or more lower extremities. For example, processor 3320 can generate a treatment regime that involves repetitively compressing the person's chest so that the chest experiences a compression phase and a recoil phase or decompression, and compressing the person's lower extremity using the compression device during at least some of the recoil phases. The compression protocol can be administered at least in part by a compression regulator 3350 of system 3300, optionally via compression mechanism lines 3360.

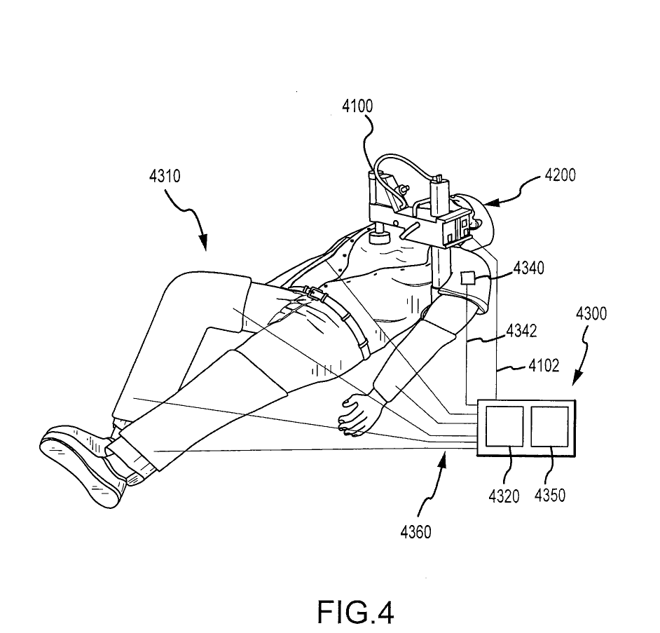

FIG. 4 illustrates a compression system 4300 according to embodiments of the present invention. Compression system 4300 can deliver compression to one or more limbs or lower extremities of patient 4200 via compression mechanisms 4310. An operator or rescuer can situate or position a stand alone compression device or automated compression apparatus 4100 at or near the patient's body. Exemplary compression devices include those such as the Thumper® (Michigan Instruments, Grand Rapids, Mich.). Compression device 4100 includes a compression element or pad that can be placed against the patient's chest. In use, the compression element applies repetitive compressions to the patient's chest. Compression system 4300 may include a computer or processor 4320 that can control operation of the compression mechanisms. Optionally, processor 4320 may control operation of the compression device 4100, for example via compression device line 4102.

Compression treatment system 4300 may include one or more sensors 4340 that can detect physiological parameters of the patient. Sensors can be configured to detect physiological parameters such as heart rate, respiration rate, temperature, blood gas (e.g. O₂, CO₂) concentration, blood pressure, blood flow, respiration gas (e.g. O₂, CO₂) concentration, cardiac output, intrathoracic pressure, intrathoracic volume, cardiac electrical activity, and the like. Processor 4320 of compression system can be configured to receive signals from sensors 4340, optionally via sensor line 4342, and process the signals according to desired processing methods. Processor 4320 may also be configured to transmit operating signals or data to one or more compression mechanisms of compression system 4300. In some cases, operating or controlling signals can be based on signals or data received from sensors 4340. In this way, processor 4320 can provide a feedback technique, whereby physiological information is sensed from the patient, and a compression treatment is applied or administered to the patient based at least in part on one or more aspects of the physiological information. According to some embodiments, processor 4320 can generate a compression regime or protocol that includes one or more treatment parameters, such as pressure, duration, timing, and the like. In some cases, the compression regime includes treatment parameters for lower extremity or limb compressions, and optionally includes treatment parameters for chest compressions. For example, processor 4320 can generate a compression protocol that involves applying compressions to one or more lower extremities of a patient during a recoil or decompression phase. Optionally, processor 4320 can generate a compression protocol that involves applying compressions to the patient's chest. In some cases, processor 4320 generate synchronized compressions of the chest and one or more lower extremities. For example, processor 4320 can generate a treatment regime that involves repetitively compressing the person's chest so that the chest experiences a compression phase and a recoil phase or decompression, and compressing the person's lower extremity using the compression device during at least some of the recoil phases. The compression protocol can be administered at least in part by a compression regulator 4350 of system 4300, optionally via compression mechanism lines 4360.

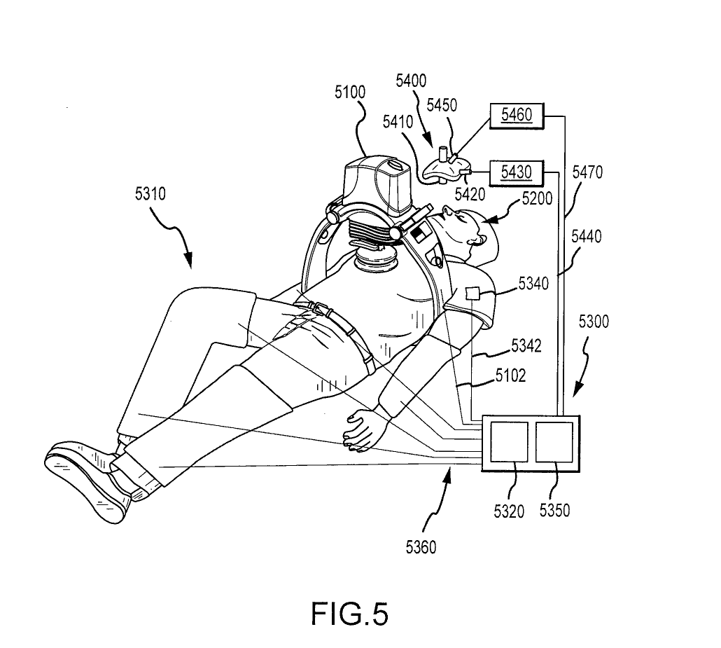

FIG. 5 illustrates a compression system 5300 according to embodiments of the present invention. Compression system 5300 can deliver compression to one or more limbs or lower extremities of patient 5200 via compression mechanisms 5310. An operator or rescuer can situate or position a stand alone Active Compression DeCompression (ACDC) device 5100 to the patient's body. Exemplary ACDC devices include those such as the LUCAS™ Chest Compression System (Jolife AB, Lund, Sweden). ACDC device 5100 includes a suction element, such as an elastomeric cup, that can be placed against the patient's chest. In use, the suction element mechanically applies compressions to the chest, and also pulls up or lifts the chest to create decompression, resulting in additional amounts of negative pressure in the thorax, thereby facilitating the process of filling the heart with blood. Compression system 5300 may include a computer or processor 5320 that can control operation of the compression mechanisms, for example via compression mechanism lines 5360. Optionally, processor 5320 may control operation of the ACDC device 5100, for example via compression device line 5102.

Compression treatment system 5300 may include one or more sensors 5340 that can detect physiological parameters of the patient. Sensors can be configured to detect physiological parameters such as heart rate, respiration rate, temperature, blood gas (e.g. O₂, CO₂) concentration, blood pressure, blood flow, respiration gas (e.g. O₂, CO₂) concentration, cardiac output, intrathoracic pressure, intrathoracic volume, cardiac electrical activity, and the like. Processor 5320 of compression system can be configured to receive signals from sensors 5340, optionally via sensor line 5342, and process the signals according to desired processing methods. Processor 5320 may also be configured to transmit operating signals or data to one or more compression mechanisms of compression system 5300. In some cases, operating or controlling signals can be based on signals or data received from sensors 5340. In this way, processor 5320 can provide a feedback technique, whereby physiological information is sensed from the patient, and a compression treatment is applied or administered to the patient based at least in part on one or more aspects of the physiological information. According to some embodiments, processor 5320 can generate a compression regime or protocol that includes one or more treatment parameters, such as pressure, duration, timing, and the like. In some cases, the compression regime includes treatment parameters for lower extremity or limb compressions, and optionally includes treatment parameters for chest compressions. For example, processor 5320 can generate a compression protocol that involves applying compressions to one or more lower extremities of a patient during a recoil or decompression phase. Optionally, processor 5320 can generate a compression protocol that involves applying compressions to the patient's chest. In some cases, processor 5320 generate synchronized compressions of the chest and one or more lower extremities. For example, processor 5320 can generate a treatment regime that involves repetitively compressing the person's chest so that the chest experiences a compression phase and a recoil phase or decompression, and compressing the person's lower extremity using the compression device during at least some of the recoil phases. The compression protocol can be administered at least in part by a compression regulator 5350 of system 5300, optionally via compression mechanism lines 5360.

As shown in FIG. 5, in some cases a mask 5400 may be used during a treatment protocol, according to embodiments of the present invention. U.S. patent application Ser. No. 12/141,864 filed Jun. 18, 2008, incorporated herein by reference, describes masks which can be used during treatments. As discussed there, systems may include a housing that defines a central lumen, and a ventilation tube 5410 having a central lumen that can be connected to or placed in fluid communication with the patient's respiratory system at its distal end. Ventilation tubes can include any patient connection or airway system having a central lumen through which respiratory gases may pass, e.g., an endrotracheal tube, laryngeal mask airway device, supraglottic airway device, nasal masks, full face masks, lipseal mouthpieces, and the like. Typically, a ventilation tube provides a connection or passage to an airway of a patient or individual. In some cases, a facial mask can have a fitting 5420 for coupling to the external vacuum source 5430, and the valve system and the fitting can be incorporated into the facial mask. Vacuum source 5430 may be in operative communication with, and in some cases controlled at least in part by, processor 5320, optionally via vacuum source line 5440. In some aspects, the external vacuum source includes an in-hospital vacuum, a suction pump, or a portable vacuum pump. According to some embodiments, oxygen may be supplied through the valve system to supplement oxygen delivery to the patient during inhalation. For example, mask 5400 may include a fitting 5450 for coupling to an external oxygen source 5460. Oxygen source 5460 may be in operative communication with, and in some cases controlled at least in part by, processor 5320, optionally via oxygen source line 5470.

Facial mask 5400 can include or be coupled with a valve system, and can be configured to be secured to a patient's face so as to cover the mouth, nose, or both. Mask 5400 and valve system are examples of equipment that in some cases may be used to lower intrathoracic pressures and thereby lower intracranial and intraocular pressures. In some cases, mask 5400 may include a valve system with a threshold valve and an airway member that interfaces with the person's airway, where the threshold valve is configured to prevent or impede respiratory gas flow to the person's lungs during a portion of an inhalation event until the inspiration equals or exceeds an opening pressure of the threshold valve. However, it will be appreciated that other valve systems and other coupling arrangements may be used. In some cases, mask 5400 may incorporate an impedance threshold device (ITD) or an intrathoracic pressure regulator (ITPR). As such the invention is not intended to be limited to the specific valve system and mask described herein.

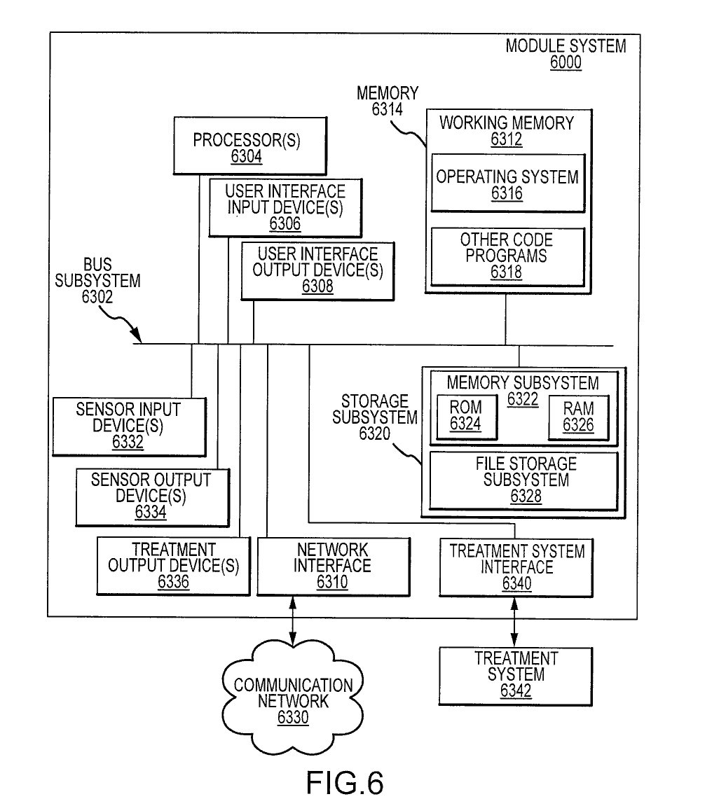

FIG. 6 is a simplified block diagram of an exemplary module system that broadly illustrates how individual system elements for a computer or module system 6000 may be implemented in a separated or more integrated manner, for example as part of a processor or treatment system according to embodiments of the present invention. Module system 6000 is well suited for detecting or monitoring physiological parameters in a patient and for controlling aspects of medical interventions or treatments administered to the patient. Module system 6000 is shown comprised of hardware elements that are electrically coupled via a bus subsystem 6302, including one or more processors 6304, one or more input devices 6306 such as user interface input devices, one or more output devices 6308 such as user interface output devices, a network interface 6310, and a treatment system interface 6340 that can receive signals from and transmit signals to treatment system 6342.

In some embodiments module system 6000 also comprises software elements, shown as being currently located within working memory 6312 of memory 6314, including an operating system 6316 and other code 6318, such as a program designed to implement methods of the invention.

Likewise, in some embodiments module system 6000 may also include a storage subsystem 6320 that can store the basic programming and data constructs that provide the functionality of the various embodiments of the present invention. For example, software modules implementing the functionality of the methods of the present invention, as described herein, may be stored in storage subsystem 6320. These software modules are generally executed by the one or more processors 6304. In a distributed environment, the software modules may be stored on a plurality of computer systems and executed by processors of the plurality of computer systems. Storage subsystem 6320 can include memory subsystem 6322 and file storage subsystem 6328. Memory subsystem 6322 may include a number of memories including a main random access memory (RAM) 6326 for storage of instructions and data during program execution and a read only memory (ROM) 6324 in which fixed instructions are stored. File storage subsystem 6328 can provide persistent (non-volatile) storage for program and data files, and may include tangible storage media which may optionally embody patient, treatment, assessment, or other data. File storage subsystem 6328 may include a hard disk drive, a floppy disk drive along with associated removable media, a Compact Digital Read Only Memory (CD-ROM) drive, an optical drive, DVD, CD-R, CD-RW, solid-state removable memory, other removable media cartridges or disks, and the like. One or more of the drives may be located at remote locations on other connected computers at other sites coupled to module system 6000. The modules implementing the functionality of the present invention may be stored by file storage subsystem 6328. In some embodiments, the software or code will provide protocol to allow the module system 6000 to communicate with communication network 6330. Often such communications will include dial-up or internet connection communications.

It is appreciated that system 6000 can be configured to carry out various methods of the present invention. For example, processor component or module 6304 can be a microprocessor control module configured to receive physiological parameter signals from sensor input device or module 6332 or user interface input device or module 6306, and to transmit treatment signals to treatment output device or module 6336, user interface output device or module 6308, network interface device or module 6310, or any combination thereof. Each of the devices or modules according to embodiments of the present invention can include one or more software modules on a computer readable medium that is processed by a processor, or hardware modules, or any combination thereof. Any of a variety of commonly used platforms, such as Windows, MacIntosh, and Unix, along with any of a variety of commonly used programming languages, may be used to implement embodiments of the present invention.

User interface input devices 6306 may include, for example, a touchpad, a keyboard, pointing devices such as a mouse, a trackball, a graphics tablet, a scanner, a joystick, a touchscreen incorporated into a display, audio input devices such as voice recognition systems, microphones, and other types of input devices. User input devices 6306 may also download a computer executable code from a tangible storage media or from communication network 6330, the code embodying any of the methods of the present invention. It will be appreciated that terminal software may be updated from time to time and downloaded to the terminal as appropriate. In general, use of the term “input device” is intended to include a variety of conventional and proprietary devices and ways to input information into module system 6000.

User interface output devices 6306 may include, for example, a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from module system 6000 to a user.

Bus subsystem 6302 provides a mechanism for letting the various components and subsystems of module system 6000 communicate with each other as intended. The various subsystems and components of module system 6000 need not be at the same physical location but may be distributed at various locations within a distributed network. Although bus subsystem 6302 is shown schematically as a single bus, alternate embodiments of the bus subsystem may utilize multiple busses.

Network interface 6310 can provide an interface to an outside network 6330 or other devices. Outside communication network 6330 can be configured to effect communications as needed or desired with other parties. It can thus receive an electronic packet from module system 6000 and transmit any information as needed or desired back to module system 6000. In addition to providing such infrastructure communications links internal to the system, the communications network system 6330 may also provide a connection to other networks such as the internet and may comprise a wired, wireless, modem, and/or other type of interfacing connection or connectivity.

It will be apparent to the skilled artisan that substantial variations may be used in accordance with specific requirements. For example, customized hardware might also be used and/or particular elements might be implemented in hardware, software (including portable software, such as applets), or both. Further, connection to other computing devices such as network input/output devices may be employed. Module terminal system 6000 itself can be of varying types including a computer terminal, a personal computer, a portable computer, a workstation, a network computer, or any other data processing system. Due to the ever-changing nature of computers and networks, the description of module system 6000 depicted in FIG. 6 is intended only as a specific example for purposes of illustrating one or more embodiments of the present invention. Many other configurations of module system 6000 are possible having more or less components than the module system depicted in FIG. 6. Any of the modules or components of module system 6000, or any combinations of such modules or components, can be coupled with, or integrated into, or otherwise configured to be in connectivity with, any of the treatment system embodiments disclosed herein. Relatedly, any of the hardware and software components discussed above can be integrated with or configured to interface with other medical assessment or treatment systems used at other locations.

In some embodiments, the module system 6000 can be configured to receive a physiological parameter of a patient at an input module. Physiological parameter data can be transmitted to an assessment module where a physiological profile is determined. The profile can be output to a system user via an output module. In some cases, the module system 6000 can determine a treatment protocol for the patient, based on a physiological parameter or profile, for example by using a treatment module. The treatment can be output to a system user via an output module. Optionally, certain aspects of the treatment can be determined by a treatment output device, and transmitted to a treatment system. Any of a variety of data related to the patient can be input into the module system, including age, weight, sex, treatment history, medical history, and the like. Parameters of treatment regimens or diagnostic evaluations can be determined based on such data.

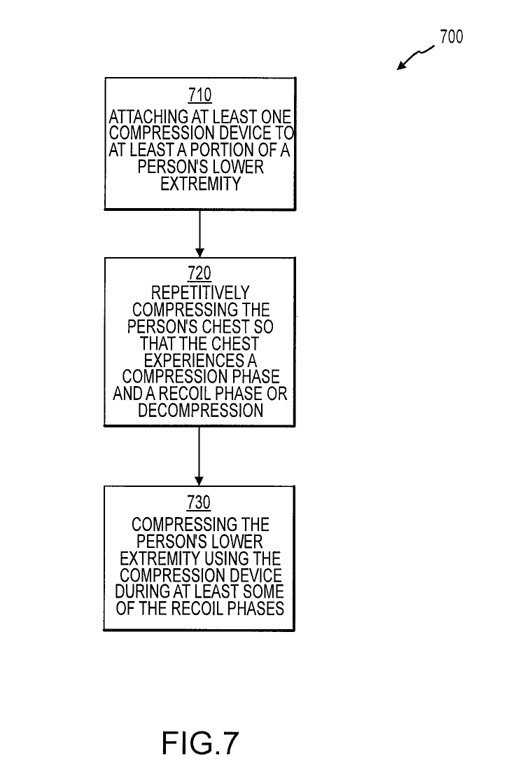

FIG. 7 illustrates aspects of a treatment method according to embodiments of the present invention. Treatment method 700 may include attaching at least one compression device to at least a portion of a person's lower extremity, as shown by step 710. Treatment method 700 may also include repetitively compressing the person's chest so that the chest experiences a compression phase and a recoil phase or decompression, as shown by step 720. Treatment method 700 may further include compressing the person's lower extremity using the compression device during at least some of the recoil phases, as shown by step 730.

The invention has now been described in detail for purposes of clarity and understanding. However, it will be appreciated that certain changes and modifications may be practiced within the scope of the appended claims.

What is claimed is:

1. A method for enhancing circulation, comprising:
attaching at least one compression device to at least a portion of a person's lower extremity;
repetitively compressing the person's chest so that the chest experiences a compression phase and a recoil phase or decompression;
compressing the person's lower extremity using the compression device during at least some of the recoil phases.

2. A method as in claim 1, wherein the chest is compressed using a circumferential vest.

3. A method as in claim 1, further comprising coupling a valve system to the person's airway, the valve system being configured to prevent respiratory gas flow to the person's lungs during the decompression phase until a negative airway pressure achieved equals the opening pressure of the valve system.

4. A method as in claim 1, further comprising withdrawing air from the person's lungs using a vacuum source until a certain negative intrathoracic pressure is achieved.

5. A method as in claim 3, wherein the opening pressure of the valve system is between about −7 cm H₂O and about −20 cm H₂O

6. A system for enhancing circulation, the system comprising:
a chest wall compression/decompression mechanism to repeatedly compress and decompress the patient's chest;
at least one lower extremity compression device that is adapted to be coupled to at least a portion of a person's lower extremity;
a controller to control actuation of the chest wall compression/decompression mechanism and the lower extremity compression device such that the lower extremity compression device compresses the person's lower extremity following at least some of the chest wall compressions.

7. A system as in claim 6, further comprising a valve system that is adapted to be coupled to the person's airway, the valve system being configured to prevent respiratory gas flow to the person's lungs during decompression until a negative airway pressure achieved equals the opening pressure of the valve system.

8. A system as in claim 7, wherein the opening pressure of the valve system is between about −7 cm H₂O and about −20 cm H₂O.

9. A method for administering circulation treatment to a person, comprising:
contacting a compression device with at least a portion of a lower extremity of the person;
repetitively compressing the person's chest so that the chest experiences a compression phase and a decompression phase;
compressing the portion of the lower extremity using the compression device during at least a portion of the decompression phase.

10. A method as in claim 9, wherein a lower extremity compression is timed to occur with each chest wall recoil.

11. A method as in claim 9, wherein a lower extremity compression is timed to occur after a certain number of chest wall recoil cycles.

12. A method as in claim 9, wherein the person is in cardiac arrest.

13. A method as in claim 9, further comprising cooling at least a second portion of the lower extremity with the compression device.

14. A method as in claim 9, further comprising interfacing a valve system to a person's airway, the valve system having a threshold valve and an airway member that interfaces with the person's airway, the threshold valve being configured to prevent or impede respiratory gas flow to the person's lungs during a portion of an inhalation event until the inspiration equals or exceeds an opening pressure of the threshold valve; and
supplying a small level of external vacuum at a location downstream of the threshold valve.

15. A method as in claim 9, wherein the chest is compressed using a compression vest.

16. A method as in claim 9, wherein the chest is compressed using an automated compression apparatus.

17. A method as in claim 9, wherein the chest is compressed using an active compression decompression device.

18. A method as in claim 9, further comprising sensing at least one physiological parameter of the person.

19. A method as in claim 18, wherein a chest compression parameter is determined based on the sensed physiological parameter.

20. A method as in claim 18, wherein a lower extremity compression parameter is determined based on the sensed physiological parameter.

21. A method as in claim 18, further comprising coordinating a chest compression and a lower extremity compression based on the sensed physiological parameter.

22. A device for administering cardiopulmonary resuscitation to or treating low circulation in a patient, comprising:
a chest compression pad configured to compress the chest;
a chest decompression mechanism configured to decompress the chest either passively or actively;
a signal processor configured to record a sensed physiological parameter; and
a lower extremity compression device that periodically compresses a lower extremity in a coordinated manner with a chest compression, a chest decompression, or both.

23. A device as in claim 22, comprising a power source that drives the chest compression pad, the chest decompression mechanism, and the lower extremity compression device, and that is coupled with a means to coordinate compressions and decompressions of the chest and the lower extremities.

24. A method to treat a patient experiencing cardiac arrest or low blood circulation, comprising:
providing to the patient a periodic positive pressure ventilation and generating a negative airway pressure when not providing the positive pressure ventilation from −3 to −15 mmHg;
actively compressing and either actively or passively decompressing the patient's chest after each compression; and
compressing a lower extremity of the patient in a synchronized manner with the chest compressions based upon a timing device or a measured physiological parameter.
